(19) United States
(12) Patent Application Publication
(10) Pub. No.: US 2026/0335917 A1
(43) Pub. Date: Oct. 29, 2026

(54) A Dispenser

(71) Applicant: Reckitt & Colman (Overseas) Hygiene Home Limited, Slough, Berkshire (GB)

(72) Inventors: Martin Richard BUTLER, Causeway Bay, Wan Chai District (HK); Guillaume KENSICHER, Hull (GB); Bin LIAO, Dongguan, Guangdong (CN); Christopher Brian WITTY, Hull (GB)

(21) Appl. No.: 19/107,336

(22) PCT Filed: Sep. 1, 2022
(86) PCT No.: PCT/CN2022/116508

§ 371 Date: Feb. 27, 2025

(51) Int. Cl. B05B 12/00; G01R 19/165

(52) U.S. Cl. CPC B05B 12/00; G01R 19/16576

(57) ABSTRACT

A liquid fragrance dispenser comprising a dispensing device and a refill bottle configured to store liquid fragrance, wherein the refill bottle comprises a body configured to form a reservoir to store the liquid fragrance and a spray head configured to moved relative to the body to activate a spray mechanism comprising a pump to dispense liquid from the reservoir; the dispensing device comprises a motor configured to move the spray head downwards to activate the pump to move the liquid fragrance from the reservoir and dispense the liquid fragrance from the spray head; the dispensing device further comprises a low voltage detector configured to determine whether a voltage is below a threshold value;

and the dispensing device is configured to stop dispensing the liquid fragrance when the low voltage detector determines that the voltage is below the threshold value.

The environmental benefit of using non-aerosol dispensers as a source of fluid has led to development in spraying fragrances and other fluids via devices from a non-aerosol fluid refill.

In general, devices for spraying fragrances, deodorising agents and sanitising fluids into a room generally consist of a device containing a removable source of fluid. In many devices, the source of fluid is an aerosol-based fluid that disperses a large distance upon activation. Spraying non-aerosol liquids requires a greater force to be used for activation because the force is directly related to the distance the liquid is projected.

The present invention relates to a device for spraying a fluid and particularly, but not exclusively to a device for spraying fluids such as fragrances, deodorizing fluids and/or a pest control material or the like in a non-aerosol form reliably and with a more uniform performance. The present invention also relates to a method of using such a device.

In an aspect of the invention, there is a liquid fragrance dispenser comprising a dispensing device and a refill bottle configured to store liquid fragrance, wherein the refill bottle comprises a body configured to form a reservoir to store the liquid fragrance and a spray head configured to moved relative to the body to activate a spray mechanism comprising a pump to dispense liquid from the reservoir; wherein the dispensing device comprises a motor configured to move the spray head down to activate the pump to move the liquid fragrance from the reservoir and dispenses the liquid fragrance from the spray head; wherein the dispensing device further comprises a low voltage detector configured to determine whether the voltage is below a threshold value; and wherein the dispensing device is configured to stop dispensing the liquid fragrance when the low voltage detector determines the voltage is below a threshold value.

Aspects of the disclosure are also described in detail, by way of example only, with reference to the accompanying drawings, in which:

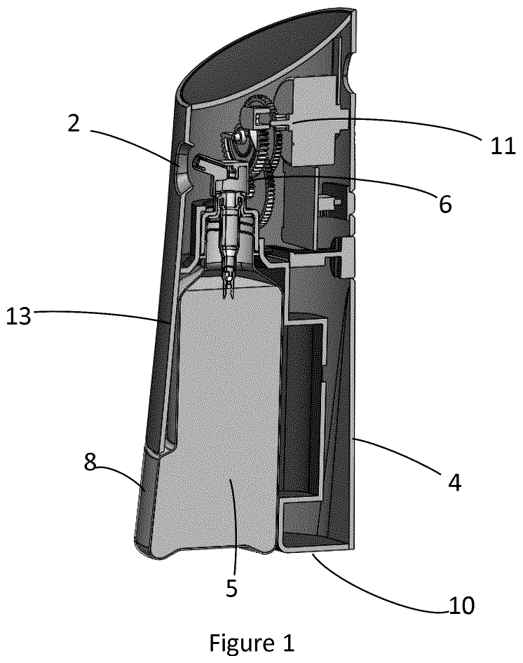

FIG. 1 shows a non-aerosol dispensing device with a refill bottle inserted into the dispensing device;

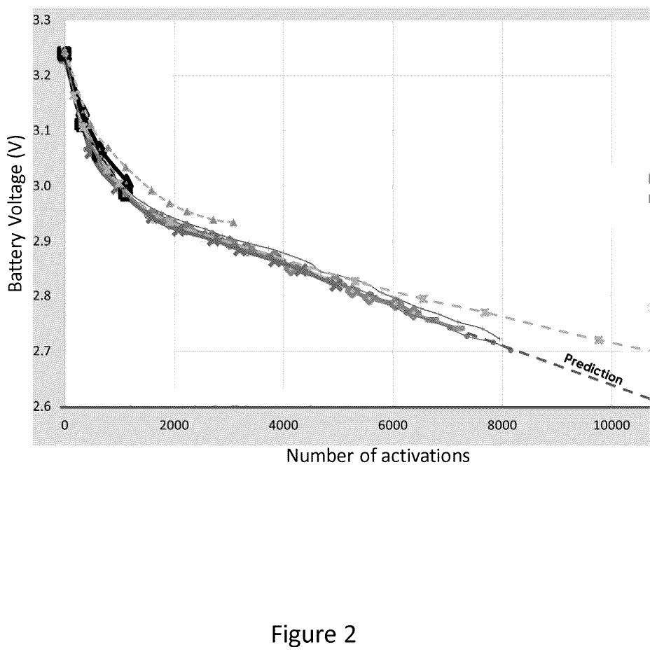

FIG. 2 shows a graph showing the decrease in the voltage of the battery with number of activations of the dispensing device;

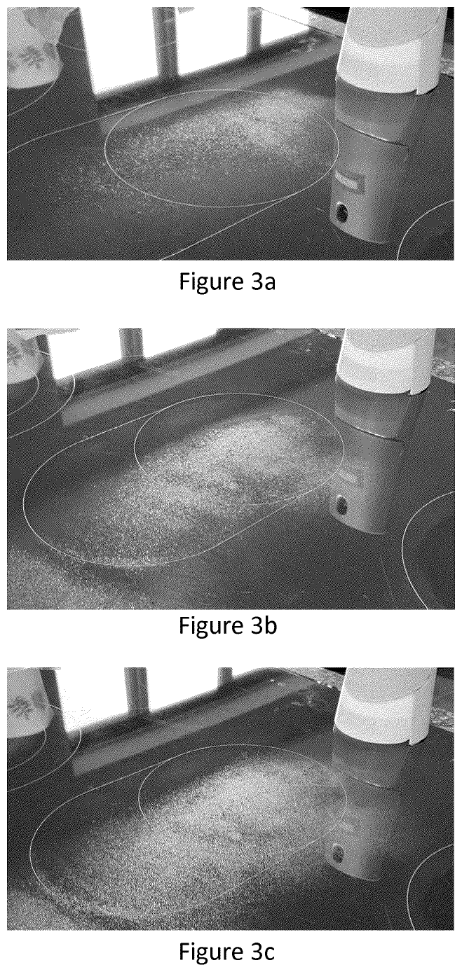

FIG. 3a shows an example of the dispensing device activation with a low fallout of fluid;

FIG. 3b shows an example of the dispensing device activation with a medium fallout of fluid;

FIG. 3c shows an example of the dispensing device activation with a high fallout of fluid; and

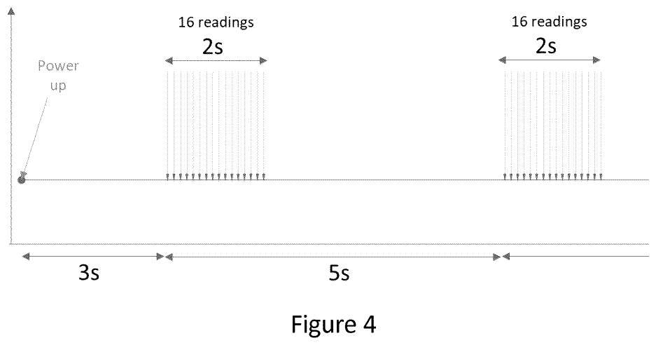

FIG. 4 shows an example of voltage sampling by a low voltage detector.

Embodiments will now be described with reference to the Figures.

FIG. 1 shows a refill bottle 8 located in a dispensing device 4. The dispensing device 4 comprises a refill receiving portion 13, a spray orifice 2 and a trigger mechanism 11. The trigger mechanism 11 comprises a motor and gearbox.

The refill bottle 8 comprises a spray head 6 and a body 5.

The refill bottle 8 is configured to hold the fluid for spraying. The fluid is held in a reservoir formed by the body 5 and configured to be dispensed via the spray head 6. In the example illustrated, the spray head 6 is coupled to the body 5 via a neck and the spray head 6 configured to moved relative to the body 5 to activate a spray mechanism to dispense liquid from the reservoir.

As illustrated in FIG. 2, the refill bottle 8 is configured to be located in the refill receiving portion 13 of the dispensing device 4. When the refill bottle 8 is located in the dispensing device 4 the spray head 6 is configured to align with the spray orifice 2 so that, in use, the spray head 6 dispenses the fluid via the spray orifice 2 when triggered by the trigger mechanism 11.

The dispensing device 4 is configured to be placed on a surface with at least part of a base 10 in contact with the surface. In the example illustrated, the base 10 has three points of contact with the surface. It is advantageous to have three points of contact to the surface to reduce instability of the dispensing device 4 when it is placed on the surface. The dispensing device 4 may also be configured to be attached to a surface, for example the dispensing device 4 may be attached to a wall.

The dispensing device 4 is configured to dispense fluid from the refill bottle 8 by imparting a force on the spray head 6 so that the spray moves down to activate a spray mechanism. For example, the spray mechanism comprises a pump coupled to the spray head that moves fluid from the reservoir and dispenses the fluid from the spray head. In this example, the motor is configured to be coupled to a gearbox. The motor and the gearbox of the dispensing device is configured to move the spray head down to activate the pump.

In an example where a non-aerosol liquid is held in the refill bottle the propulsion of the liquid from the spray head is related to the velocity that is applied to the spray head by the dispensing device. For example, the dispensing device moving the spray head with a larger velocity will lead to the liquid being projected from the spray head at a faster speed and the liquid will travel further and/or with increased from the dispensing device.

The force applied by the motor of the dispensing device is dependent upon power available to the motor. In the example described above, the motor of the dispensing device 4 is configured to be powered by a battery. The voltage provided by the battery may decrease as the remaining power in the battery decreases. The reduction in voltage may lead to a decrease in performance of the dispensing device.

FIG. 2 shows the change in the voltage from the battery of the dispensing device 4 with the number of activations of the dispensing device 4. In the example illustrated, the voltage drops initially at a faster rate with the number of activations and then more slowly after around 2000 activations.

FIGS. 3a, 3b and 3c show the impact of the voltage on spray performance. These Figures show fallout of liquid after activating the spray of the dispensing device. The level of fallout is based on the amount of liquid that is dispensed at a distance close to the dispensing device. The images shown in FIGS. 3a-3c illustrate the fallout with the white liquid proximate the dispensing device.

FIG. 3a shows the fallout of liquid from an example of a dispensing device 4 with a battery voltage of 3.23V. FIG. 3b shows the fallout of liquid from an example of a dispensing device 4 with a battery voltage of 2.588 V. FIG. 3c shows the fallout of liquid from an example of a dispensing device 4 with a battery voltage of 2.426V.

The images show the level of fallout, corresponding to the increase in liquid proximate to the dispensing device, increasing as the voltage of the battery decreases. A high level of fallout from the device leads to an unacceptable level of performance. For example, a high level of fallout means a smaller amount of liquid will be projected into the room and the efficacy of the dispensing device reduced.

The dispensing device comprises a microprocessor unit (MCU) and a low voltage detector. The low voltage detector is configured to be coupled to the battery via the MCU and measure the voltage from the battery. The low voltage detector measures the voltage from the battery and determines whether the voltage from the battery is below a threshold value. In an example, the threshold level of the voltage may be based on the voltage of the battery that leads to a fallout from the dispensing device that gives an acceptable level of performance and therefore efficacy of the dispensing device. The threshold level of the voltage may also be based on minimum number of spray activations during the lifetime of the battery.

The dispensing device is programmed to set the voltage threshold. For example, the MCU senses the voltage of the battery and the low voltage detector determines whether or not the voltage is above the threshold value.

In the example described, the dispensing device is configured to stop dispensing liquid when the low voltage detector determines the battery voltage is below a threshold value. For example, the dispensing device stops the motor from activating and when the low voltage detector determines the battery voltage is below a threshold value.

In an example, the low voltage detector is activated a short time after the initial power up of the dispensing device. For example, the low voltage detector is activated 2 to 7 seconds after power up, for example 3 seconds after power up. The low voltage detector determines whether the voltage is above or below the threshold periodically. For example, the low voltage detector determines whether the voltage is above or below the threshold every 2 to 10 seconds, for example every 5 seconds.

The low voltage detector is configured to sample the voltage numerous times over a period of time to determine whether the voltage is a steady state. FIG. 4 shows an example of the sampling undertaken by the low voltage detector. The numerous samples of the voltage over a period of time ensures that the low voltage detector can determine whether the voltage is above or below the threshold value when the motor is activated during the sampling period. In the example illustrated in FIG. 4, the low voltage detector may sample the voltage by executing 16 voltage checks over a 2 second period. In this example, the low voltage detector samples the voltage every 5 seconds.

In an example, the dispensing device comprises two 1.5V AA batteries. In this example, the threshold of the low voltage detector may be between 2.5 V and 2.7V, for example, 2.55V to 2.65V, for example 2.58V to 2.62V. In an example, the threshold of the low voltage detector may be 83% to 90% of the total voltage of a battery which powers the dispensing device, for example 85% to 88% of the total voltage of the battery, for example 86% to 87% of the total voltage of the battery.

In an example, when the low voltage detector determines that the voltage is below the threshold value the dispensing device requires a reset to restart. For example, removing the battery from the dispensing device would reset the device. The requirement to reset the dispensing device to restart prevents unstable performance resulting from fluctuations in battery voltage resulting from the battery voltage slowly recovering.

In the example described above, the MCU of the dispensing device is configured to cease to provide a spray signal once the low voltage detector determines the voltage is below the threshold value.

In the examples described, the dispensing device dispenses a liquid fragrance. The dispensing device may also dispense deodorizing fluids and/or a pest control material or the like.

Further modifications and developments can be made without departing from the scope of the invention described herein.

1. A liquid fragrance dispenser comprising a dispensing device and a refill bottle configured to store liquid fragrance, wherein:
the refill bottle comprises a body configured to form a reservoir to store the liquid fragrance and a spray head configured to moved relative to the body to activate a spray mechanism comprising a pump to dispense liquid from the reservoir;
the dispensing device comprises a motor configured to move the spray head downwards to activate the pump to move the liquid fragrance from the reservoir and dispense the liquid fragrance from the spray head;
the dispensing device further comprises a low voltage detector configured to determine whether a voltage is below a threshold value; and
the dispensing device is configured to stop dispensing the liquid fragrance when the low voltage detector determines that the voltage is below the threshold value.

2. The liquid fragrance dispenser of claim 1, wherein:
the dispensing device further comprises a microprocessor unit and the microprocessor unit is configured to cease to provide a spray signal once the low voltage detector determines that the voltage is below the threshold value.

3. The liquid fragrance dispenser of claim 1, wherein the low voltage detector is configured to determine whether the voltage is below a threshold value by executing 16 voltage checks over a 2 second period.

4. The liquid fragrance dispenser of claim 1, wherein the low voltage detector is configured to determine whether the voltage is below a threshold value every 5 seconds.

5. The liquid fragrance dispenser of claim 1, wherein the low voltage detector is activated 2 to 7 seconds after power up.

6. The liquid fragrance dispenser according to claim 1, wherein the threshold of the low voltage detector is between 83% and 90% of the total voltage of a battery which powers the dispensing device.

7. The liquid fragrance dispenser of according to claim 1, wherein the dispensing device is configured to require a reset to restart dispensing when the low voltage detector determines that the voltage is below the threshold value.

8. The liquid fragrance dispenser of claim 1, wherein the low voltage detector is activated 3 seconds after power up.

9. The liquid fragrance dispenser according to claim 1, wherein the threshold of the low voltage detector is between 85% and 88% of the total voltage of a battery which powers the dispensing device.

10. The liquid fragrance dispenser according to claim 1, wherein the threshold of the low voltage detector is between 86% and 87% of the total voltage of a battery which powers the dispensing device.
